(19) United States
(12) Patent Application Publication
(10) Pub. No.: US 2020/0315698 A1
(43) Pub. Date: Oct. 8, 2020

(54) ENERGY TREATMENT INSTRUMENT

(71) Applicant: OLYMPUS CORPORATION, Tokyo (JP)

(72) Inventor: Yuji SAKAI, Kodaira-shi (JP)

(73) Assignee: OLYMPUS CORPORATION, Tokyo (JP)

(21) Appl. No.: 16/910,206

(22) Filed: Jun. 24, 2020

Related U.S. Application Data
(63) Continuation of application No. PCT/JP2018/035354, filed on Sep. 25, 2018.

(30) Foreign Application Priority Data
Dec. 25, 2017 (JP) 2017-248381

(51) Int. Cl. A61B 18/14; A61B 34/20; A61B 17/24

(52) U.S. Cl. CPC A61B 18/1477; A61B 34/20; A61B 2017/00734; A61B 2034/2063; A61B 2034/2055; A61B 17/24

(57) ABSTRACT

An energy treatment instrument includes: an insertion section in which. a longitudinal axis is defined, a probe, an output section, and a detector. The probe is provided on a distal side along the longitudinal axis of the insertion section and is configured to be pierced into a biological tissue. The output section is provided on the probe and is configured to output energy to an outside of the probe when supplied with energy. The detector is configured to recognize presence of an organ which is located farther than an end portion of the probe, when the probe is pierced into the biological tissue.

### CROSS-REFERENCE TO RELATED APPLICATIONS

This application. is a Continuation Application of PCT Application No. PCT/JP2018/035354, filed Sep. 25, 2018 and based upon and claiming the benefit of priority from prior Japanese Patent Application No. 2017-248381, filed Dec. 25, 2017, the entire contents of all of which are incorporated herein by reference.

### FIELD

The present invention relates to an energy treatment instrument.

### BACKGROUND

For example, U.S. Pat. No. 5,823,197 discloses a treatment instrument in which a needle-like probe is provided on an energy delivery device. The treatment instrument can output energy from the probe into a mucous membrane of the inferior turbinate, in a state in. which the probe is pierced in the inferior turbinate.

### BRIEF SUMMARY OF THE INVENTION

According to one aspect of the present invention, an energy treatment instrument includes: an insertion section in which a longitudinal axis is defined, a probe, an output section, and a detector. The probe is provided on a distal side along the longitudinal axis of the insertion section and is configured to be pierced into a biological tissue. The output section is provided on the probe and is configured to output energy to an outside of the probe when supplied with energy. The detector is configured to recognize presence of an organ which is located farther than an end portion of the probe, when the probe is pierced into the biological tissue.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic view illustrating a treatment system according to each of first to third embodiments including modifications;

FIG. 2 is a view illustrating a cross section of an end effector and a distal portion of an insertion section in a state in which a needle section and guide rods are protected by a cover portion, and a schematic configuration of a handle, in an energy treatment instrument of the treatment system according to the first embodiment;

FIG. 3 is a view illustrating a cross section of the end effector and the distal portion of the insertion section in a state in which the needle section and guide rods are exposed from the cover portion, and a schematic configuration of the handle, in the energy treatment instrument according to the first embodiment;

FIG. 4 is a schematic view of the end effector and the distal portion of the insertion section of the energy treatment instrument, as viewed in a direction of an arrow IV in FIG. 2 and FIG. 3;

FIG. 5 is a schematic view illustrating a probe of the needle section and an energy output section provided in the probe, and illustrating a denaturing region of a biological tissue at a. time when. energy is output from the energy output section;

FIG. 6A is a schematic view illustrating a state in which the end effector and insertion section of the energy treatment instrument according to the first embodiment are inserted through the external nostril, vestibulum nasi and inferior meatus, and the end effector is abutted on the posterior nasal nerve of a treatment target in the cavitas nasi;

FIG. 6B is a schematic view which is an enlarged view of a part of FIG. 6A, and illustrates a state in which the insertion section of the energy treatment instrument is disposed in the inferior meatus, and the end effector is abutted on the posterior nasal nerve of the treatment target in the cavitas nasi;

FIG. 6C is a schematic cross-sectional view illustrating a state in which distal ends of the guide rods shown in FIG. 3 are put in contact with a bone (hard tissue) on the rear side of the inferior turbinate, and the energy output sections are disposed near, or put in contact with, the posterior nasal nerve on the rear side of the inferior turbinate;

FIG. 7 is a schematic view illustrating a denaturing region of a biological tissue between an electrode provided on the guide rod and the energy output section provided in the probe of the needle section in. an end effector of an energy treatment instrument according to a first modification of the first embodiment;

FIG. 8 is a schematic view illustrating a state in which the needle section is protected by the cover portion, when viewed in a direction of arrow VIII in FIG. 9, in an energy treatment instrument according to a second modification of the first embodiment;

FIG. 9 is a schematic view illustrating a state in which the needle section is protected by the cover portion, when viewed in a direction of arrow IX in FIG. 8, in the energy treatment instrument according to the second modification of the first embodiment;

FIG. 10 is a schematic view illustrating a state in which the needle section is exposed from the cover portion, when viewed in a direction of arrow X in FIG. 11, in the energy treatment instrument according to the second modification of the first embodiment;

FIG. 11 is a schematic view illustrating a state in which the needle section is exposed from the cover portion, when viewed in a direction of arrow XI in FIG. 10, in the energy treatment instrument according to the second modification of the first embodiment;

FIG. 12 is a schematic view illustrating a state in which the needle section is protected by the cover portion, when viewed. in a direction of arrow XII in FIG. 13, in an energy treatment instrument according to a third modification of the first embodiment;

FIG. 13 is a schematic view illustrating a state in which the needle section is protected by the cover portion, when viewed in a direction of arrow XIII in FIG. 12, in the energy treatment instrument according to the third modification of the first embodiment;

FIG. 14 is a schematic view illustrating a state in which the needle section. is exposed from the cover portion, when viewed in a direction of arrow XIV in FIG. 15, in the energy treatment instrument according to the third modification of the first embodiment;

FIG. 15 is a schematic view illustrating a state in which the needle section is exposed from the cover portion, when viewed in a direction of arrow XV in FIG. 14, in the energy treatment instrument according to the third modification of the first embodiment;

FIG. 16 is a schematic cross-sectional view illustrating a state in which the energy output sections are disposed near, or put in contact with, the posterior nasal nerve on the rear side of the inferior turbinate, while a position of a blood vessel (or a position of a bone) in which a blood flow exists is being confirmed by using a sensor, in an energy treatment instrument according to a second embodiment;

FIG. 17 is a schematic view illustrating a method in which a blood flow is confirmed by using the sensor of the energy treatment instrument according to the third embodiment; and

FIG. 18 is a schematic cross-sectional view illustrating a state in which the energy output sections are disposed near, or put in contact with, the posterior nasal nerve on the rear side of the inferior turbinate, while a position of a blood vessel (or a position of a bone) in which a blood flow exists is being confirmed by using a sensor, in an energy treatment instrument according to a third embodiment.

### DETAILED DESCRIPTION

Hereinafter, embodiments for carrying out the present invention will be described with reference to the accompanying drawings.

### First Embodiment

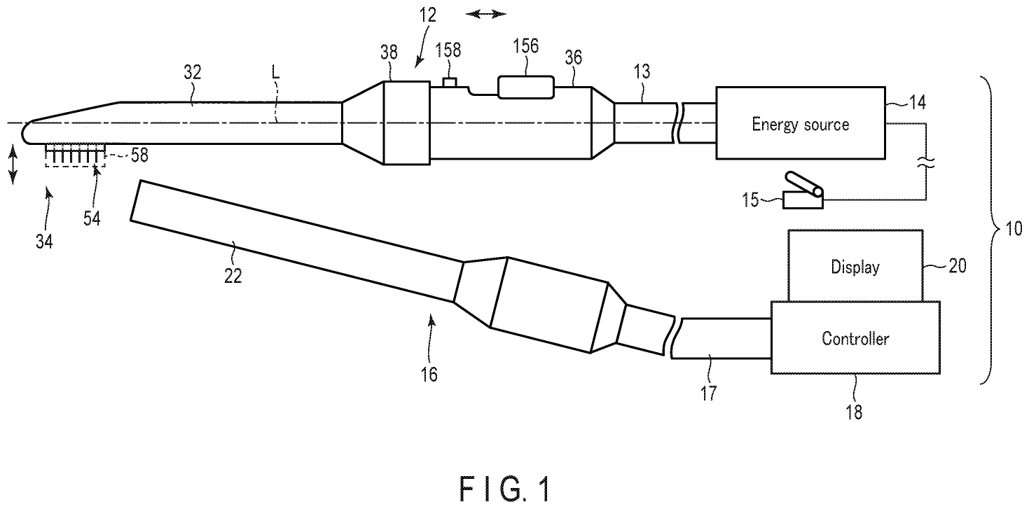

A first embodiment will be described with reference to FIG. 1 to FIG. 6C.

A treatment system 10 illustrated in FIG. 1 is used for treating the cavitas nasi, in particular, the inferior turbinate. The treatment system 10 includes an energy treatment instrument 12 and an energy source 14. The treatment system 10 of the embodiment includes an endoscope 16 and a controller 18 having a function as a power source, in addition to the energy treatment instrument 12 and energy source 14. The controller 18 includes, for example, a processor. The endoscope 16 is controlled by the processor of the controller 18. As the treatment system 10, the endoscope 16 and controller 18 are not necessarily required.

Electric power is supplied to the endoscope 16 via a cable 17 from the controller 18 which is used, for example, as a power source. The endoscope 16 captures an image of a position opposed to, for example, a distal end of an insertion section 22, and the captured image is displayed on a display 20. The insertion section 22 of the endoscope 16 may be formed to such rigidity as to as to keep the shape of the insertion section 22, or may be formed have such flexibility as to allow bending as appropriate.

The energy treatment instrument 12 includes an insertion section 32 in which a longitudinal axis L is defined, and an end effector (treatment section) 34. The energy treatment instrument 12 includes a handle 36 which is provided on a proximal side of the insertion section 32. A proximal portion of the insertion section 32 is coupled to the handle 36 which is grasped by a surgeon.

It is preferable that the energy treatment instruction 12 includes a rotary knob (rotary section) 38 which is inserted between the insertion section 32 and the handle 36 and is rotatable around the longitudinal axis L of the insertion section 32. The rotary knob 38 may be provided on the insertion section 32.

It is preferable that the rotary knob 38 is formed as one piece with the insertion section 32. When the rotary knob 38 is rotated relative to the handle 36 around the longitudinal axis L of the insertion section 32, the insertion section 32 is rotated relative to the handle 36 in the same direction as the rotational direction of the rotary knob 38 around the longitudinal axis L of the insertion section 32.

The energy source 14 is connected to the handle 36 via a cable 13. A switch 15 is connected to the energy source 14. Aside from being connected to the energy source 14, the switch 15 may be provided, for example, on the insertion section 32, handle 36 or rotary knob 38.

When the switch 15 is pushed, energy is transmitted from the energy source 14 to an energy output section (output section) 56 through a base 52 (to be described later) of the end effector 34. In the present embodiment, the energy output section 56 is described as being a high-frequency electrode through which high-frequency current is passed, but various kinds of energy may be used for treatment, such as by using a heater which generates heat. In addition, the energy output section 56 may be configured such that a heater is disposed on the high-frequency electrode and, thereby, the energy treatment instrument 12 can simultaneously perform a treatment using high-frequency current and a treatment using the heat of the heater.

The energy source 14 may be provided on the handle 36. In this case, energy is transmitted from the energy source 14, which includes a battery (not shown) provided in the handle 36, to the energy output section (output section) 56 through the insertion section 32 and the base 52 (to be described later) of the end effector 34.

The switch 15 may be configured to transmit a signal to the energy source 14 in interlock with the position of a cover portion (cover) 58 (to be described later), the position of a moving rod 82 (to be described later), and/or the position of an operation element 156 (to be described later). For example, when the operation element 156 is positioned in a first end portion 154a of a. slot 154 (to be described later) and the cover portion 58 is located in a first position (to be described later), energy may be prevented from being transmitted from the energy source 14 to the end effector 34 even when the switch 15 is operated. In addition, when the operation element 156 is positioned in a second end portion 154b of the slot 154 and the cover portion 58 is located in a second position (to be described later), energy may be transmitted from the energy source 14 to the end effector 34 by the operation of the switch 15.

The end effector 34 is provided on a distal portion of the insertion section 32. In the present embodiment, the insertion section 32 and the end effector 34 are formed as one piece. Thus, the insertion section 32 and end effector 34 can rotate as one piece, relative to the handle 36, in a manner to follow the operation of the rotary knob 38 around the longitudinal axis L.

A lock, which prevents an unintended rotation of the insertion section 32 relative to the handle 36, may be provided between the handle 36 and the rotary knob 38, or between the handle 36 and the insertion section 32. The lock may be implemented by applying frictional force between the handle 36 and rotary knob 38 or between the handle 36 and insertion section 32, or may be implemented by providing a mechanism which prevents a movement between the handle 36 and rotary knob 38 or between the handle 36 and insertion section 32, such as by engaging the handle 36 and rotary knob 38 or the handle 36 and insertion section 32. For example, a mechanism may be provided which prevents a rotation between the handle 36 and rotary knob 38 or between the handle 36 and insertion section 32 in interlock with the pressing of the switch 15 and/or the movement of the operation element 156 which keeps the state in which the cover portion 58 is in a second position (to be described later).

The insertion section 32 is formed, for example, in a pipe shape. The insertion section 32 may be formed of a proper material. For example, a metallic material, which has an outer peripheral surface coated with an electrically insulating material, is used for the insertion section 32.

A so-called malleable material, which can be bent in a proper shape by a load of pressure, may be used for that portion of the insertion section 32, which is located at a position between a proximal end of the end effector 34 and the handle 36. The insertion section 32 is bent before a treatment, for example, in accordance with the shape of the nose of each patient, and the bent shape can be kept during the treatment. Thus, in the state in which the end effector 34 on the distal side of the insertion section 32 is set in a proper direction relative to the insertion section 32, the end effector 34 can keep its direction during the treatment.

Since the end effector 34 and insertion section 32 are inserted in the patient's narrow cavity, there is a case in which proper external force acts on the end effector 34 and insertion section 32 from a wall surface in the cavitas nasi. In such a case, the insertion section 32, which is formed of the malleable material, have proper resistance to the external force from, for example, the wall surface in the body cavity. Thus, although the insertion section 32 allows proper bending, the insertion section 32 is prevented from being suddenly bent to a large degree. Accordingly, the end effector 34 on the distal side of the insertion section 32 can keep the state in which the end effector 34 is set in the proper direction relative to the insertion section 32.

Besides, it is preferable that the insertion section 32 includes a bending section having a similar structure to a bending section of an insertion section of a publicly known endoscope that can be inserted into, for example, the stomach or large intestine. In this case, the end effector 34 may be made to access the vicinity of a treatment target while properly and actively bending the bending section in accordance with the shape in the cavitas nasi. In addition, by properly bending the bending section, the end effector 34 on the distal side of the insertion section 32 is set in a proper direction relative to the insertion section 32, and this state is kept. Note that, for example, the handle 36 may be provided with a lock which prevents an unintended rotation of the bending section of the insertion section 32 during the treatment. The lock may operate in interlock with, for example, the pressing of the switch 15 and/or the movement of the operation element 156 which keeps the state in which the cover portion 58 is in a second position (to be described later). The lock may be interlocked with the above-described mechanism which prevents a rotation between the handle 36 and rotary knob 38 or between the handle 36 and insertion section 32.

In the present embodiment, the base 52, needle section 54, output section 56 and cover portion 58 of the end effector 34 are preferably pointed in a direction deviating from the longitudinal axis L of the insertion section 32 (i.e. a direction crossing the longitudinal axis L). In this embodiment, for the purpose of simple description, it is assumed that the end effector 34 is pointed in a direction perpendicular to the longitudinal axis L. The direction in which the end effector 34 is set relative to the insertion section 32 is not limited to the direction perpendicular to the longitudinal axis L, but may be set as appropriate.

A housing 50 of the end effector 34 is provided on the distal side of the insertion section 32. It is preferable that the housing 50 is formed as one piece with the distal portion of the insertion section 32. Thus, like the insertion section 32, it is preferable that the housing 50 has an outer peripheral surface coated with an electrically insulating material. It is preferable that a distal end 51 of the housing 50 is formed in an obtuse shape. Thus, when the distal end 51 of the housing 50 is passed through a passage from the entrance toward the treatment target, the passage is protected.

The end effector 34 includes a base (energy delivery device) 52 formed in a plate shape or block shape, a needle section 54, and an energy output section 56 provided in the needle section 54. In the present embodiment, the end effector 34 includes a cover portion 58 which protects the needle section 54 in the passage from the entrance toward the treatment target. The cover portion 58 is provided outside the base 52 and is movable relative to the base 52. By the movement in a predetermined direction, the cover portion 58 can advance and retreat (can move) between a first position in which the cover portion 58 protects a biological tissue from a tip end 104 (to be described later) and energy output section 56 of the needle section 54, and a second position in which the tip end 104 and energy output section 56 of the needle section 54 can treat the biological tissue. In the present embodiment, it is preferable that the cover portion 58 is movable in a direction crossing the longitudinal axis L, in particular, in a direction perpendicular to the longitudinal axis L.

It is preferable that the cover portion 58 is formed of an electrically insulating resin material. Although it is preferable that the cover portion 58 is provided in the housing 50, the cover portion 58 is not necessarily required. In the present embodiment, a description is given of an example in which the cover portion 58 is provided outside the needle section 54 (probes 102 to be described later).

The housing 50 includes a recess-shaped inner peripheral surface (recessed surface) 50a. The base 52 is provided on the distal side along the longitudinal axis L of the insertion section 32, and is fixed to the housing 50. specifically, the base 52 is fixed to the inner peripheral surface 50a of the housing 50. The needle section 54 is supported on that side of the base 52, which is opposite to the part fixed to the inner peripheral surface 50a of the housing 50. The needle section 54 refers to a region where a bundle of a plurality of needle-shaped probes (micro-needles) 102 (to be described later), or a plurality of probes 102 are included.

The insertion section 32 is provided with at least a pair of electrical paths (transmission paths). The base 52 is electrically connected to the energy source 14 via lead wires (not shown) or a structural member or the like of the insertion section 32. For example, the insertion section 32 is electrically connected to the energy output sections 56, and may be used as a part of the transmission path for transmitting energy which is supplied from the energy source 14 to the energy output sections 56. For example, a moving rod 82 provided inside an inner peripheral surface 32a of the insertion section 32 is electrically connected to the energy output sections 56, and may be used as a part of the transmission path for transmitting energy which is supplied from the energy source 14 to the energy output sections 56. In addition, the base 52 is used as a delivery device which transmits energy to the energy output sections 56 provided in the needle section 54.

The needle section 54 includes a plurality of probes 102. The probes 102 are provided on the distal side along the longitudinal axis L of the insertion section 32. Each probe 102 projects in a first direction relative to the base 52, and can be pierced into a biological tissue. Each probe 102 includes a tip end (needle point) 104 at its distal end. Thus, the needle section 54 includes tip ends 104 projecting from the base 52, and is pierced into the biological tissue from the tip ends 104.

Each probe 102 includes a basal portion 106 which is supported on the base 52. In the present embodiment, it is preferable that, between the basal portion 106 and tip end 104 of each probe 102, a portion excluding the energy output section 56 has electrical insulation properties.

It is preferable that the respective probes 102 are straight and parallel with each other. The extending direction of each probe 102 agrees with the direction (first direction) in which the end effector 34 is directed. The extending direction (first direction) of each probe 102 is, for example, perpendicular or substantially perpendicular to the longitudinal axis L. The extending direction of each probe 102 is not limited to the direction perpendicular or substantially perpendicular to the longitudinal axis L, and may be set as appropriate. Although each probe 102 is preferably formed to be straight from the basal portion 106 to tip end 104, as described above, each probe 102 may be bent as appropriate.

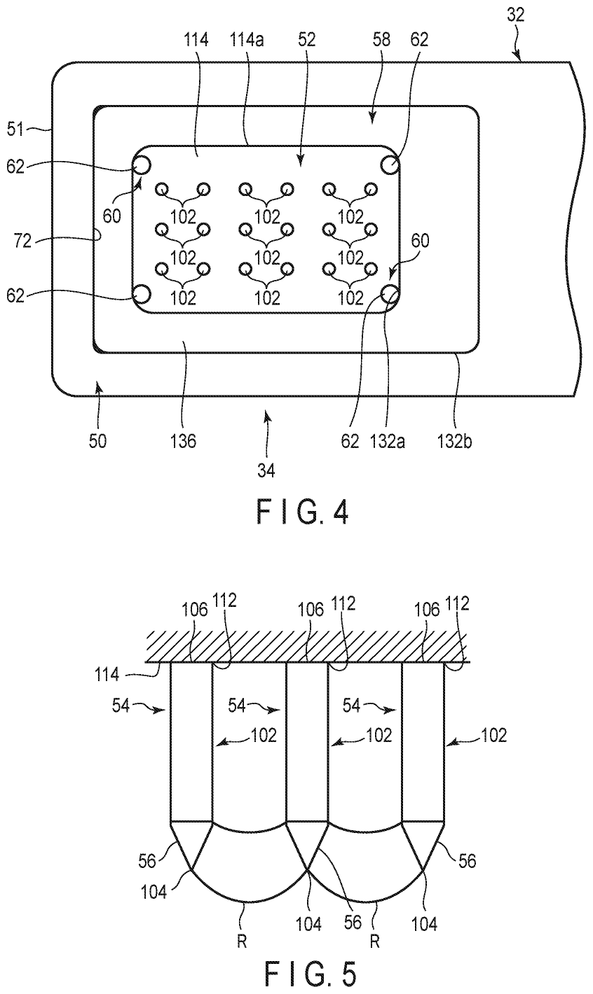

The base 52 includes a support portion 112 which supports the basal portions 106 of the probes 102 of the needle section 54. As illustrated in FIG. 4, in the support portion 112, for example, the probes 102 are arranged in a lattice shape at proper intervals. The density of probes 102 in the needle section 54 is properly set, for example, based on the size of a treatment region (a region indicated by sign R in FIG. 5) in the energy output section 56 of each probe 102. The base 52 includes a defining surface 114 which defines a length (projection length) of each probe 102 of the needle section 54 between the tip end 104 of the probe 102 and the defining surface 114 of the base 52. For the purpose of simple description, in the present embodiment, it is assumed that the defining surface 114 is a planar surface and the extending direction of each probe 102 is perpendicular to the defining surface 114.

It is preferable that the lengths (projection lengths) of the respective probes (micro-needles) 102 relative to the defining surface 114 of the base 52 are identical or substantially identical. The magnitudes of the length, diameter, etc. of each probe 102 are set based on a material and a treatment target, and also the position of a second end face (end portion) 136 of the cover portion 58. For example, it is preferable that the outside diameter of each probe 102 is about 200 μm. It is preferable that the length of each probe 102 is set such that, for example, a piercing depth from the surface of a soft tissue T (e.g. a mucosal epithelial layer) (to be described later) to the energy output section 56 is about 0.05 mm to 0.8 mm, although the length of each probe 102 depends on the positional relationship with the cover portion 58.

The end effector 34 of the present embodiment includes a detector (sensing portion) 60 which detects the presence of an organ which is located farther than a tip end (end portion) 104 of the probe 102, when the probes 102 of the needle section 54 are pierced into a soft tissue (biological tissue) T. The detector 60 includes four guide rods 62 in the present embodiment. The guide rods 62 are juxtaposed with the probes 102. The guide rods 62 are formed of an electrically insulating material. In the present embodiment, as illustrated in FIG. 4, an example is illustrated in which the guide rods 62 are disposed in an outside region of the needle section 54. However, the guide rods 62 may be disposed in an inside region of the needle section 54, for example, among the probes 102.

In the present embodiment, the detector 60 is described as including four guide rods 62. However, the number of guide rods 62 may not be plural, such as four, but may be only one.

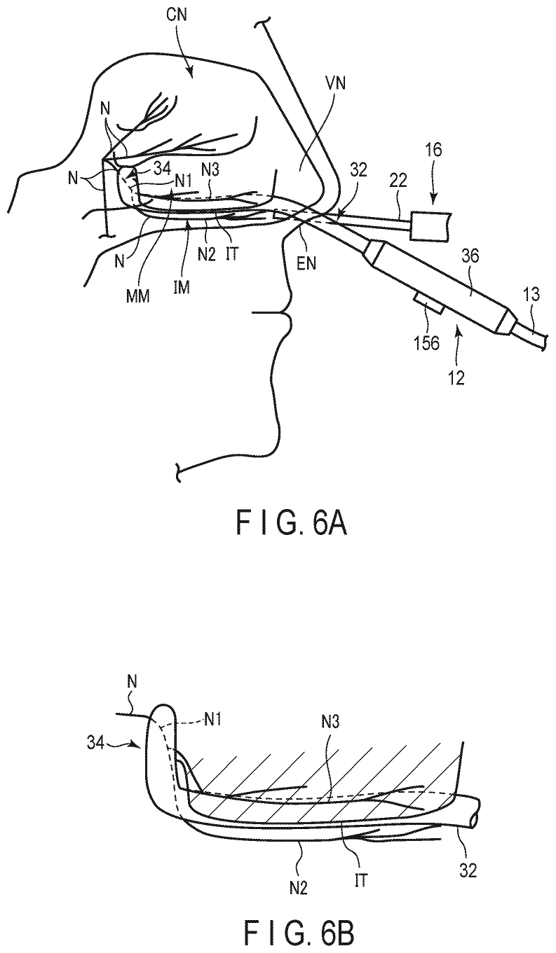

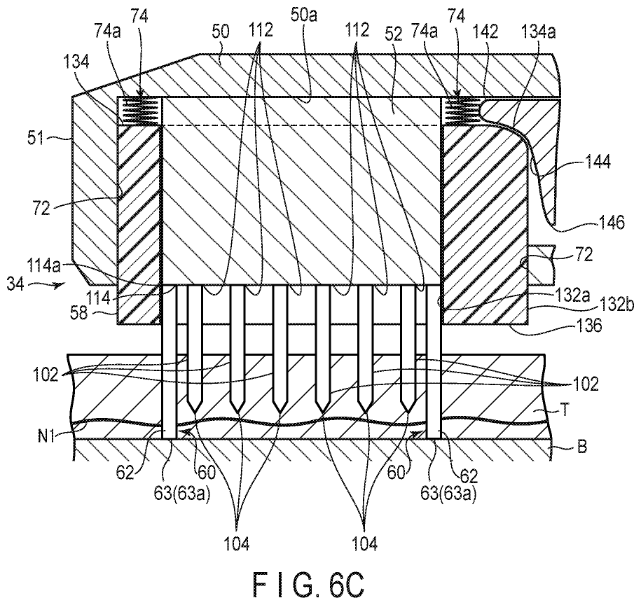

A distal end 63 of each guide rod 62 can be abutted on a hard tissue such as a bone B (see FIG. 6C), for example. In the present embodiment, the distal end 63 of the guide rod 62 is farther from the base 52 than the tip end 104 of the probe 102. The distal end 63 of the guide rod 62 is abutted on the bone B, and thereby used as a sensor which enables the surgeon to understand the position of the bone B. Specifically, that the distal end 63 of the guide rod 62 comes in contact with the organ such as the bone B earlier than the tip end 104 of the probe 102 of the needle section 54 is one kind of detector 60. Note that the positional relationship between the distal end 63 of the guide rod 62 and the tip end 104 of the probe 102 is set such that when the distal end 63 of the guide rod 62 is in contact with the bone B, the tip end 104 of the probe 102 is disposed at a position where a nerve N near the surface of the bone B exists.

It is preferable that a protection member 63a which protects the bone B is fixed to the distal end 63 of the guide rod 62. It is preferable that the protection member 63a is formed of an electrically insulating resin material or rubber material. The protection member 63a can protect not only the bone B but also the nerve N and blood vessel V existing near the bone B.

The housing 50 includes a guide 72 which guides the movement of the cover portion 58 relative to the base 52 and needle section 54. The guide 72 is provided outside the base 52. Thus, the cover portion 58 is provided outside the base 52. In this embodiment, the cover portion 58 is movable in a direction parallel to or substantially parallel to the extending direction of the probes 102 which extend straight from the base 52. The extending direction of the probes 102 is a direction crossing the longitudinal axis L of the insertion section 32. The cover portion 58 is movable in a direction crossing the longitudinal axis L of the insertion section 32. In other words, the second end face (end portion) 136 of the cover portion 58 is movable in the direction in which the needle section 54 projects. Accordingly, in the housing 50, the cover portion 58, which is provided on the outer periphery of the base 52 and needle section 54, is supported so as to be movable relative to the housing 50, base 52 and needle section 54.

The end effector 34 includes an urging body 74 which can move the cover portion 58 along the guide 72 relative to the base 52 and needle section 54. For example, the urging body 74 is provided between the cover portion 58 and the inner peripheral surface 50a of the housing 50.

In the present embodiment, for example, a plurality of coil springs 74a are used as the urging body 74. One end of each coil spring 74a is supported on the inner peripheral surface 50a of the housing 50. The other end of each coil spring 74a is supported on a first end face (end edge) 134 (to be described later) of the cover portion 58. The coil spring 74a of this embodiment urges the first end face 134 of the cover portion 58 such that the first end face 134 approaches the inner peripheral surface 50a of the housing 50. As the urging body 74, a rubber member may be sued in place of the coil springs 74a.

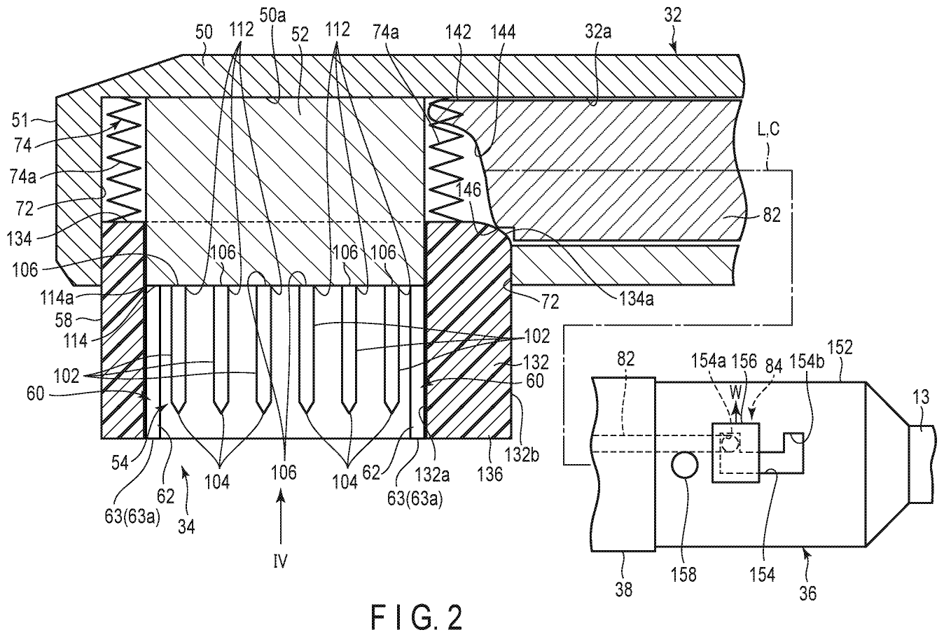

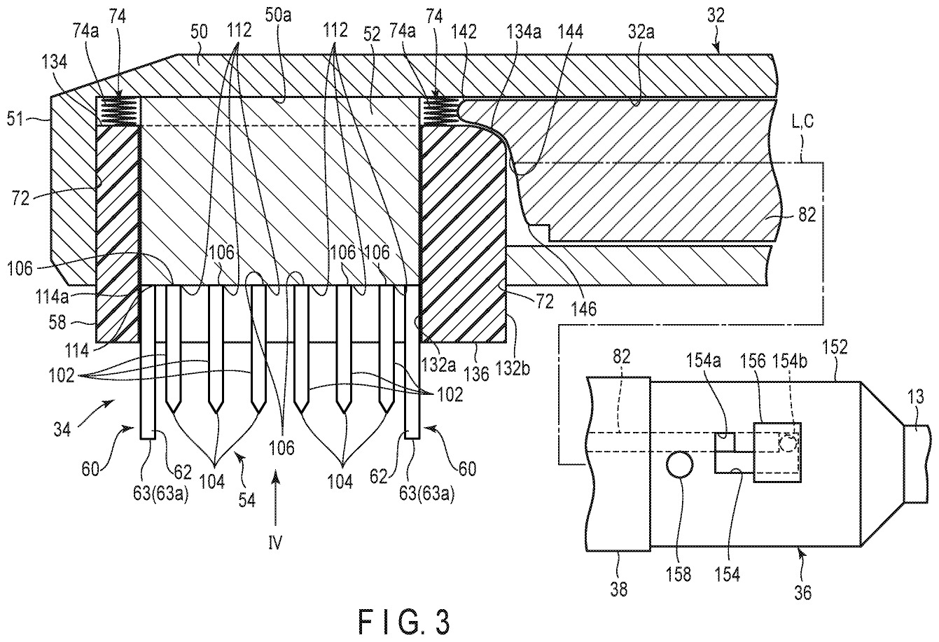

In the present embodiment, the energy treatment instrument 12 includes a lock mechanism 84 which is provided on the handle 36 and holds the position of the cover portion 58 relative to the base 52. In the lock mechanism 84, for example, the operation element 156 is held by being disposed in either of end portions 154a and 154b of a slot 154. Thus, in the energy treatment instrument 12 of this embodiment, the cover portion 58 is locked in a first position illustrated in FIG. 2, and is locked in a second position illustrated in FIG. 3. Hence, in this embodiment, the lock mechanism 84 is provided on the handle 36, and holds the position of the cover portion 58 relative to the base 52 and needle section 54.

The lock mechanism 84 may be formed on the insertion section 32 in which the moving rod 82 is included. Thus, it should suffice if the lock mechanism 84 is provided on at least one of the handle 36 and moving rod 82.

In the present embodiment, the cover portion 58 is provided outside an outer edge 114a of the defining surface 114. The cover portion 58 includes a cover body 132. In the present embodiment, the cover body 132 is described as having a cylindrical shape (an annular-shaped transverse cross section). However, the shape of the cover body 132 is not limited to the cylindrical shape, and the cover body 132 may have some other proper transverse cross-sectional shape such as a substantially C-shape or U-shape.

The cover body 132 has an inner peripheral surface (wall surface) 132a and an outer peripheral surface (wall surface) 132b. The cover body 132 has a first end face 134 which is opposed to the inner peripheral surface 50a on the depth side of the housing 50, and a second end face (a reference surface or a reference edge) 136 which is located on the opposite side to the first end face 134. The first end face 134 and second end face 136 may be planar surfaces or curved surfaces. The second end face 136 can advance and retreat (can move) in a predetermined direction (specifically, the direction of projection of the needle section 54) relative to the base 52, and is used as a reference surface (reference edge) which defines a positional relationship with the tip ends 104 of the probes 102 of the needle section 54. The second end face 136 of the cover portion 58 is formed in the direction in which the needles section 54 projects relative to the defining surface 114 of the base 52, and is formed to be continuous with distal ends of the inner peripheral surface 132a and outer peripheral surface 132b of the cover body 132.

As illustrated in FIG. 2, the second end face 136 of the cover portion 58 is disposed in a position (on the projection direction side) which is equal to the tip end 104 of the needle section 54 or projects from the tip end 104. This position is defined as “first position”. Thus, when the cover portion 58 is in the first position, the second end face 136 of the cover portion 58 is disposed in a position which is equal to the tip end 104 of the probe 102 of the needle section 54, or in a position which is farther from the defining surface 114 of the base 52 than the tip end 104. In this manner, when the cover portion 58 is in the first position, the second end face (end portion) 136 of the cover portion 58 is disposed in a position which is equal to the tip end 104 of the needle section 54, or in a position which projects from the tip end 104, thereby protecting the tip ends 104 and output sections 56 of the needle section 54. Specifically, when the cover portion 58 is in the first position, the cover portion 58 surrounds and covers the outside of the needle section 54 in such a state that the tip ends (needle points) 104 of the probes 102 are not exposed.

As illustrated in FIG. 3, the second end face 136 of the cover portion 58 is disposed in a position (on the base 52 side) which is closer to the base 52 than the tip ends 104 and energy output sections 56 of the needle section 54. This position is defined as “second osition”. Thus, the second end face 136 of the cover portion 58 is disposed in a position which is closer to the defining surface 114 of the base 52 than the tip ends 104 and energy output sections 56 of the probes 102 of the needle section 54. In this manner, when the cover portion 58 is in the second position, the second end face (end portion) 136 of the cover portion 58 is evacuated from the tip ends 104 and output sections 56 of the needle section 54. In other words, when the cover portion 58 is in the second position, the tip ends (needle points) 104 and energy output sections 56 of the probes 102 project from the second end face (end portion) 136 with respect to the cover portion 58. At this time, the tip ends (needle points) 104 and energy output sections 56 of the probes 102 are exposed from the cover portion 58. Thus, when the cover portion 58 is in the second position, the end effector 34 is capable of piercing the tip ends 104 and energy output sections 56 of the needle section 54 into the biological tissue.

In this manner, the cover portion 58 can advance and retreat (can move) between the first position (see FIG. 2) and the second position (see FIG. 3). Thus, when the cover portion 58 moves from the first position to the second position, the tip ends (needle points) 104 and energy output sections 56 of the probes 102 can project from the second end face (end portion) 136 of the cover portion 58.

In the present embodiment, in the second position where the tip ends 104 of the probes 102 are exposed, the second end face 136 of the cover portion 58 is located closer to the tip ends 104 of the needle section 54 than the defining surface 114 of the base 52. Although not illustrated, in the second position where the tip ends 104 of the probes 102 are exposed, the second end face 136 of the cover portion 58 may be located farther from the tip ends 104 of the needle section 54 than the defining surface 114 of the base 52.

As illustrated in FIG. 5, the energy output section 56 is provided in a predetermined position between the tip end 104 and base portion 106 of each probe 102. In the present embodiment, the energy output section 56 is disposed in the tip end 104 or near the tip end 104. The energy output section 56 can output energy to the outside of the probe 102 of the needle section 54 by being supplied with energy from the energy source 14 via the cable 13. Here, the energy output sections 56 provided in neighboring probes 102 are used as electrodes of different polarities. The energy output sections 56 provided in the neighboring probes 102 are electrically connected to the energy source 14 via different electrical paths (lead wires). Note that, in each probe 102 of the needle section 54, a part at a position outside the energy output section 56 has electrical insulation properties. In addition, high-frequency current is passed via a biological tissue between the energy output sections 56 provided in the neighboring probes 102. Thus, a tissue of a region R shown in FIG. 5, for example, is locally denatured (coagulated) by the output of energy between the energy output sections 56 of the neighboring probes 102.

The energy source 14 can monitor the state of a tissue by acquiring information of impedance or the like of the tissue near each energy output section 56, by using each energy output section 56 as a sensor. Thus, by using the present system 10, the coagulation state and/or cauterization state of the tissue can be understood as in the publicly known art. In addition, by monitoring the information of impedance or the like, the energy source 14 can recognize whether the energy output section 56 is in contact with a biological tissue. It is thus preferable that the energy source 14 outputs energy of such a degree as not to affect the biological tissue which is in contact with the energy output section 56, immediately before outputting such energy as to affect the biological tissue which is in contact with the energy output section 56. Therefore, the energy source 14 can output such energy as to affect the biological tissue which is in contact with the energy output section 56, after judging whether the energy output section 56 of each probe 102 is exactly in contact with the biological tissue.

The energy source 14 may be capable of adjusting electric current which is passed through each energy output section 56, based on biological information of each of the individual energy output sections 56.

In the present embodiment, as illustrated in FIG. 3, when the cover portion 58 is in the second position, the second end face 136 of the cover portion 58 is positioned closer to the tip ends 104 of the probes 102 than the defining surface 114 of the base 52. Thus, when the cover portion 58 is in the second position, the end effector 34 is capable of piercing the tip ends 104 and energy output sections 56 of the needle section 54 into the biological tissue. In addition, the position of the energy output section 56 relative to the second end face 136 of the cover portion 58 at a time when the cover portion 58 is in the second position corresponds to a distance for reaching the lamina propria mucosae through the mucosal epithelial layer in the soft tissue T. Furthermore, the position of the energy output section 56 relative to the second end face 136 of the cover portion 58 at a time when the cover portion 58 is in the second position corresponds to a distance for reaching a deep part of the soft tissue T, i.e. the nerve Ni near the bone B.

As described above, depending on the state of design, there may be a case in which when the cover portion 58 is in the second position, the defining surface 114 of the base 52 is closer to the tip ends 104 of the probes 102 than the second end face 136 of the cover portion 58. In this case, the projection length of the energy output section 56 relative to the defining surface 114 of the base 52 at a time when the cover portion 58 is in the second position is a distance for reaching a deep part of the soft tissue T, i.e. the nerve N1 near the bone B, through the surface of the soft tissue T.

In the present embodiment, the first end face 134 of the cover portion 58 includes an inclined surface 134a. The inclined surface 134a is formed at a position on the proximal side along the longitudinal axis L in the first end face 134 of the cover portion 58. The inclined surface 134a is formed as a planar surface or a curved surface.

In this embodiment, the energy treatment instrument 12 includes the moving rod (moving body) 82 which is provided between the handle 36 and end effector 34 and is movable along the longitudinal axis L. The moving rod 82 is formed of, for example, the same malleable material as the insertion section 32, and is bent in a manner to follow the movement of the insertion section 32.

A distal portion of the moving rod 82 includes a projection portion 142 which abuts on, or approaches, the base 52; an inclined surface 144 which is continuous with the projection portion 142; and an abutment surface 146 which is continuous with the inclined surface 144 and is abutted on the inclined surface 134a of the cover portion 58. It is preferable that the moving rod 82 and projection portion 142 are electrically insulated, for example, by applying coatings with electrical insulation properties to outer surfaces thereof.

The inclined surface 144 is formed as a planar surface or a curved surface. The inclined surface 144 is inclined to a plane perpendicular to the longitudinal axis L. The projection portion 142 is located at a position close to the inner peripheral surface 50a in the housing 50, relative to the inclined surface 144.

A proximal portion of the moving rod 82 extends to the handle 36. The handle 36 includes a main body 152 which is formed, for example, in a cylindrical shape; the slot (groove) 154 formed in the main body 152; and the operation element 156 coupled to the proximal portion of the moving rod 82 through the slot 154. The slot 154 is formed along the longitudinal axis L. The slot 154 is provided with, as parts of the lock mechanism 84, a first end portion 154a and a second end portion 154b which are continuous. The first end portion 154a is formed in a position on the distal side along the longitudinal axis L in the slot 154. The second end portion 154b is formed in a position on the proximal side along the longitudinal axis L in the slot 154. The slot 154 including the first end portion 154a and second end portion 154b is formed, for example, as a substantially U-shape as a whole. The operation element 156 is disposed in the first end portion 154a of the slot 154, when the cover portion 58 is kept, i.e. locked, in the first position illustrated in FIG. 2. The operation element 156 is disposed in the second end portion 154b of the slot 154, when the cover portion 58 is kept, i.e. locked, in the second position illustrated in FIG. 3.

As illustrated in FIG. 2, when the cover portion 58 is in the first position, it is preferable that the operation element 156 of the handle 36 is located at one end 154a of the slot 154 and is urged in a width direction W which is perpendicular to the longitudinal axis L. The abutment surface 146 of the distal portion of the moving rod 82 is abutted on the inclined surface 134a of the first end face 134 of the cover portion 58. Thus, the first end face 134 of the cover portion 58 is located apart from the inner peripheral surface 50a of the housing 50.

As illustrated in FIG. 3, when the cover portion 58 is in the second position, it is preferable that the operation element 156 of the handle 36 is located at the other end 154b of the slot 154 and is urged in the width direction W which is perpendicular to the longitudinal axis L. The inclined surface 144 of the distal portion of the moving rod 82 is abutted on the inclined surface 134a of the first end face 134 of the cover portion 58. Thus, the first end face 134 of the cover portion 58 is located near the inner peripheral surface 50a of the housing 50.

An indicator 158 is provided on the handle 36. In the present embodiment, for example, the indicator 158 emits light, only while energy from the energy source 14 is being output. In particular, it is preferable that the indicator 158 emits light, only while energy is being supplied to each energy output section 56 and a biological tissue is being treated by each energy output section 56.

Next, the function of the treatment system 10 according to the present embodiment will be described. In particular, a description will be given of an example in which a part of the posterior nasal nerve N in the soft tissue T of the nose is treated by using the energy treatment instrument 12. Note that, aside from the treatment of a part of the posterior nasal nerve N, a treatment may also be performed in other cases by using the energy treatment instrument 12.

For instance, allergic rhinitis is a disease of many patients worldwide. This disease includes seasonal allergic rhinitis generally called “hay fever”, and year-round allergic rhinitis with house dust or a pet being an allergen. Main symptoms of the allergic rhinitis include nasal congestion, snivel, sneezing, and itching. The first choice of a treatment for each symptom is, basically, medication, but an operative therapy may be applicable to a patient with a serious illness.

There are various operative therapies, which include, for example, (1) an operative therapy aiming at reducing and denaturing a mucous membrane of the nose, (2) corrective surgery of nasal cavity which aims at improving the degree of nasal airflow, and (3) an operative therapy aiming at shutting off neurotransmission.

Here, for example, such a case is mainly described that the (3) operative therapy aiming at shutting off neurotransmission is performed by using the energy treatment instrument 12. At present, it has begun to be recognized that the posterior nasal nerve induces a nasal allergic reaction and causes sniveling. As regards the sniveling due to allergic rhinitis, it has begun to be understood that a treatment of properly denaturing the posterior nasal nerve, such as by coagulation, is effective.

A surgeon sets the cover portion 58 of the end effector 34 of the energy treatment instrument 12 in the first position (see FIG. 2) in advance. As regards the posterior nasal nerve N shown in FIG. 6A, a part thereof at a position indicated by sign N1, immediately preceding a point where the nerve N branches and extends into the inferior turbinate IT, is cauterized and cut, or denatured, and thereby signal transmission from nerves N2 and N3 (see FIG. 6A and FIG. 6B), which branch and extend from the position of sign N1, can be shut off. In this case, the surgeon needs to make the end effector 34 approach the rear side of the inferior turbinate IT. Thus, the surgeon properly bends in advance the insertion section 32 on the proximal side of the end effector 34, and adjusts the direction of the end effector 34 relative to the insertion section 32. For example, the end effector 34 is bent in a substantially L-shape, relative to the insertion section 32. In this state, as illustrated in FIG. 6A, the surgeon inserts the end effector 34 of the energy treatment instrument 12 into the rear side of the inferior turbinate IT of the treatment target in the cavitas nasi ON through the patient's external nostril EN, vestibulum nasi VN and, for example, inferior meatus IM. The surgeon disposes, when necessary, a distal end of the insertion section 22 of the endoscope 16 at a position where a part of the inner wall of the cavitas nasi ON and a part of the end effector 34 of the energy treatment instrument 12 can be observed. When the endoscope 16 is used, a part of the inner wall of the cavitas nasi ON and a part of the end effector 34 of the energy treatment instrument 12 are displayed on the display 20 by the endoscope 16.

Then, the surgeon puts the second end face 136 of the cover portion 58 of the end effector 34, which is properly bent relative to the insertion section 32, into contact with the soft tissue T in which the posterior nasal nerve N1 exists. Note that the posterior nasal nerve N1 on the rear side of the inferior turbinate IT may be accessed via not the inferior meatus IM but the middle meatus MM.

Here, as illustrated in FIG. 2, in the end effector 34 of the energy treatment instrument 12 according to the present embodiment, the tip end 104 of each probe 102 is positioned closer to the defining surface 114 of the base 52 than the second end face 136 of the cover portion 58. Thus, when the end effector 34 of the energy treatment instrument 12 is inserted from the cavitas nasi ON toward the posterior nasal nerve N1 of the treatment target, the tip end 104 of each probe 102 is prevented from abutting on the wall surface in the range from the cavitas nasi ON of the patient to the posterior nasal nerve N1 of the treatment target. In addition, since the tip end 104 of each probe 102 is prevented from abutting on the wall surface in the range from the cavitas nasi ON of the patient to the posterior nasal nerve N1 of the treatment target, a load is prevented from acting on each probe 102 before the treatment target is treated.

As illustrated in FIG. 60, the posterior nasal nerve N1 of the treatment target exists, for example, along the surface of the bone B in the soft tissue T. When the surgeon puts the second end face 136 of the cover portion 58 into contact with the surface of the soft tissue T, it is preferable that the tip end 104 of each probe 102 of the needle section 54 is not in contact with the surface of the soft tissue T, but the tip end 104 of each probe 102 may be in contact with the surface of the soft tissue T.

When the surgeon puts the second end face 136 of the cover portion 58 into contact with the surface of the soft tissue T, it is preferable that the distal end 63 (protection member 63a) of the guide rod 62 is not in contact with the surface of the soft tissue T, but the distal end 63 (protection member 63a) of the guide rod 62 may be in contact with the surface of the soft tissue T.

The surgeon moves the operation element 156 of the handle 36 from the position shown in FIG. 2 to the position shown in FIG. 3, against the urging force applied to the operation element 156. At this time, in interlock with the . movement of the operation element 156, the moving rod 82 is moved toward the proximal side along the longitudinal axis L of the insertion section 32. By the urging force of the springs 74a of the urging body 74, the first end face 134 of the cover portion 58 is moved to approach the inner peripheral surface 50a of the housing 50 along the guide 72, while keeping the state in which the inclined surface 134a abuts on the inclined surface 144 of the distal portion of the moving rod 82.

When the operation element 156 is disposed in the other end 154b of the slot 154, the position of the cover portion 58 and moving rod 82 relative to the housing 50 is defined. Accordingly, the cover portion 58 of the end effector 34 is held in the second position. At this time, the projection length up to the distal end 63 (protection member 63a) of the guide rod 62 and the tip end 104 of the probe 102 relative to the second end face 136 of the cover portion 58 is defined. Thus, the projection length up to the energy output section 56 relative to the second end face 136 of the cover portion 58 is defined.

When the cover portion 58 is moving from the first position to the second position, if the distal end 63 (protection member 63a) of the guide rod 62 comes in contact with the bone B, the second end face 136 of the cover portion 58 is separated from the surface of the soft tissue T. At this time, it can be estimated that the tip end 104 of the probe 102, i.e. the energy output portion 56, is in contact with the nerve N near the surface of the bone B, or in close proximity to the nerve N.

In this state, if the surgeon pushes the switch 15, the indicator 158 provided on the handle 36 is turned on, and high-frequency current is supplied from the energy source 14 to a part at a position near the bone B in the soft tissue T through an area between the energy output sections 56 provided in the neighboring probes 102. The soft tissue T, which is in contact with each energy output section 56, and the posterior nasal nerve N1 that is a peripheral tissue thereof are denatured, for example, by being locally cauterized by high-frequency current. Note that a treatment region R (see FIG. 5) of each probe 102 in the needle section 54 is changed based on the state of the tissue, the magnitude of current, etc. Thus, a part of the soft tissue T and a part of the posterior nasal nerve N1 are cauterized. The posterior nasal nerve N1 is continuous with the nerves N2 and N3 which branch and extend. Accordingly, if the posterior nasal nerve N1 is denatured (coagulated), for example, the signal transmission from the brain to the nerve N2, N3 is shut off.

Here, the energy output section 56 is used as a sensor which acquires biological information such as impedance. In addition, the energy source 14, which is electrically connected to the energy output section 56, fully recognizes the state (biological information) of the deep part in the soft tissue T, which is in contact with the energy output section 56, and a peripheral region thereof, i.e. the posterior nasal nerve N1 and a peripheral part thereof. Thus, while high-frequency current is being passed, the state of denaturing of the tissue, which is in contact with the energy output section 56, and the posterior nasal nerve N1 in the peripheral region thereof is estimated.

In addition, when the energy source 14 judges that the biological information has reached a predetermined threshold, the energy source 14 automatically stops the output of energy to the tissue, which is in contact with the energy output section 56, and the peripheral tissue thereof. At this time, even in the state in which the switch 15 is pressed, the energy source 14 turns off the indicator 158. The surgeon can recognize, by the turn-off of the indicator 158, the end of the treatment, i.e. the end of the output of energy to the tissue, which is in contact with the energy output section 56, and the peripheral tissue thereof. Note that when the indicator 158 is turned off, the energy source 14 may completely stop the supply of energy or may pass such a weak level of current as not to affect the biological tissue. Specifically, when the indicator 158 is turned off, the output from the energy source 14 is automatically reduced.

As described above, the energy output section 56, which has reached the position near the bone B, cauterizes a portion of the deep part in the soft tissue T and a part of the posterior nasal nerve N1 by passing high-frequency current. On the other hand, the region that is coagulated and treated by the energy output section 56 is limited to a narrow range, and such a cauterizing treatment as to cause damage on the surface of the mucosal epithelial layer or the lamina propria mucosa of the soft tissue T is prevented from being performed.

By performing such treatment, a portion of the deep part in the soft tissue T and a part of the posterior nasal nerve N1 are denatured (coagulated) without causing damage on the surface of the soft tissue T. Thus, the signal transmission from the brain, to the posterior nasal nerve N1 is shut off. Furthermore, after the treatment, since the transmission of a signal from the brain to the nerves N2 and N3, into which the posterior nasal nerve N1 is branched, is shut off, the occurrence of rhinorrhea is suppressed.

When the surgeon largely moves the end effector 34, as in the case of pulling out the end effector 34 from the cavitas nasi CN, the surgeon moves the cover portion 58 from the second position to the first position. The surgeon moves the operation element 156 from the other end 154a toward the one end 154a of the slot 154. At this time, the moving rod 82 advances along the longitudinal axis L of the insertion section 32. The inclined surface 144 of the moving rod 82 pushes the inclined surface 134a of the first end face 134 of the cover portion 58, against the urging force of the springs 74a of the urging body 74. In addition, the abutment surface 146 is abutted on the inclined surface 134a. Thus, the second end face 136 of the cover portion 58 is positioned farther from the defining surface 114 of the base 52 than the tip ends 104 of the probes 102. In other words, the tip ends 104 of the probes 102 and the distal ends 63 (protection members 63a) of the guide rods 62 are positioned closer to the defining surface 114 of the base 52 than the second end face 136 of the cover portion 58. Further, the operation element 156 is kept in the state in which the operation element 156 is disposed in the one end 154a of the slot 154. Thus, the cover portion 58 is restored from the second position shown in FIG. 3 to the first position shown in FIG. 2.

As described above, when the posterior nasal nerve N1 is to be accessed by the end effector 34, it is possible that the end effector 34 is passed through the middle meatus MM, as well as the inferior meatus IM, in accordance with the state of, for example, the inferior turbinate IT. Thus, there may be a case in which the surgeon wishes to adjust the direction of the end effector 34. In this case, since the surgeon needs to operate, for example, the operation element 156, there may be a case in which it is preferable that the grasping state of the handle 36 is maintained. Thus, the surgeon rotates the rotary knob 38 around the longitudinal axis L relative to the handle 36. In accordance with the rotation of the rotary knob 38, the insertion section (cylindrical body) 32, which is coupled to the rotary knob 38, and the moving rod 82 in. the inside of the insertion section 32 are rotated together. Accordingly, the direction of the end effector 34 on the distal side of the insertion section 32 is adjusted in a proper state.

In addition, the operation element 156 of the handle 36 is moved to a proper position, and a portion of the deep part in the soft tissue T and a part of the posterior nasal nerve N1 are coagulated as needed, in the same manner as described above.

In the present embodiment, the description was given on the assumption that the insertion section 32 is bent in a substantially L-shape prior to the treatment, and the direction of the end effector 34 is adjusted. However, the insertion section 32 may be bent in advance in the substantially L-shape, and the direction of the end effector 34 may be adjusted.

Furthermore, in this embodiment, the example was described in which the nerve N1 as the treatment target is cauterized. The nerves N2 and N3 exist, for example, along the surface of the bone B in the soft tissue T. Thus, like the case in which the nerve N1 is the treatment target, the branched nerve N2, N3 may be cauterized and treated.

According to the energy treatment instrument 12 of the present embodiment, the following can be said.

By using the energy treatment instrument 12 according to the present embodiment and abutting the distal ends 63 of the guide rods 62 on the surface of the bone B, the energy output sections 56 can be disposed at positions separate from the surface of the bone B by a proper distance. Thus, in the end effector 34, the energy output section 56 provided in the probe 102 can be positioned in contact with, or in close proximity to, the nerve N1, N2, N3, which exists in the deep part of the soft tissue T and extends near the surface of the bone B. Accordingly, the treatment instrument 12 can effectively treat the nerve N1, N2, N3, by using energy. In this manner, by using the energy treatment instrument 12 of the present embodiment, the surgeon can easily and properly treat a treatment target existing at a proper depth. Therefore, according to the present embodiment, there can be provided the energy treatment instrument 12 which can easily recognize the presence of a biological tissue other than a treatment target to which energy is output.

In addition, without causing damage on the surface of the soft tissue T or casing damage on a tissue in a region at a distance from the part where the nerves N1, N2 and N3 extend, the surgeon can make the energy output section 56 of the end effector 34 of the treatment instrument 12 approach, with no invasion or low invasion, the nerve N1,

N2, N3 near the bone B, which is considered to be effective in modern treatment. In addition, the surgeon. can. denature only a part of the soft tissue T, in which the nerve N1, N2, N3 exists, and a part of the nerve N1, N2, N3, by using the energy treatment instrument 12. Specifically, a part of the soft tissue T and a part of the nerve N (N1, N2, N3) near the bone B can be cauterized by using high-frequency current. Accordingly, with use of the treatment instrument 12, the surgeon can surely cauterize and cut a part of the soft tissue T in the region, in which the nerve N1, N2, N3 exists, and a part of the nerve N1 near the bone B, by using high-frequency current in the energy output section 56. Therefore, the occurrence of rhinorrhea can be suppressed by using the energy treatment instrument 12 according to the present embodiment.

In the treatment instrument 12 according to the present embodiment, the direction of the end effector 34 can be adjusted by properly bending the insertion section 32. Thus, the end effector 34 can easily access a proper treatment target such as the nerve N2 or N3, as well as the nerve N1 on the rear side of the inferior turbinate IT.

In the treatment instrument 12 according to the present embodiment, the insertion section 32 can properly be rotated relative to the handle 36. Thus, the end effector 34 can easily access a proper treatment target such as the nerve N2 or N3, as well as the nerve N1 on the rear side of the inferior turbinate IT.

When the end effector 34 is made to access the treatment target, even if each probe 102 of the needle section 54 exists in the end effector 34, the tip end 104 of each probe 102 and the distal ends 63 of the guide rods 62 can be protected by the cover portion 58, and the path to the treatment target can be protected. In addition, when the end effector 34 is made to access the treatment target, the probes 102 of the needle section 54 can be protected by the cover portion 58.

Accordingly, by using the energy treatment instrument 12, the end effector 34 of the treatment instrument 12 can be made to access, in particular, the inside of the cavitas nasi CN that is complex and narrow, such as the inferior turbinate IT having a wide range and a projecting shape, without causing damage to other tissues.

The example was described in which the urging body 74 of the present embodiment urges the first end face 134 of the cover portion 58 such that the first end face 134 approaches the inner peripheral surface 50a of the housing 50. Alternatively, as the urging body 74, a coil spring or rubber member may be used, which urges the first end face 1.34 of the cover portion 58 such that the first end face 134 moves away from the inner peripheral surface 50a of the housing 50. At this time, if the second end face 136 of the cover portion 58 is pushed, the cover portion 58 can move from the first position to the second position against the urging force of the urging body 74. If the state in which the second end face 136 of the cover portion 58 is in contact with the surface of the soft tissue T can be maintained when a treatment is performed, it may be unnecessary to move the cover portion 58 between the first position and second position by the moving rod 82.

(First Modification)

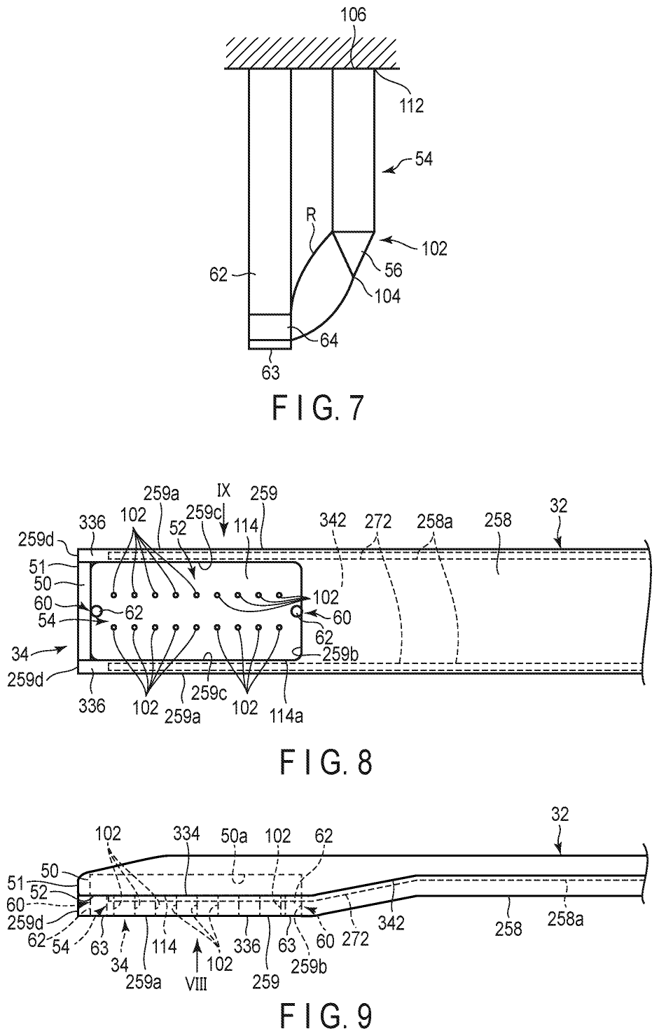

Here, referring to FIG. 7, a modification of the energy output section 56 and the guide rod 62 will be described. In the example illustrated in FIG. 5, the description was given on the assumption that when an electric current is passed from the energy source 14, the energy output sections 56 provided in the neighboring probes 102 are used as electrodes of different polarities. In the example illustrated in FIG. 7, the guide rod 62 includes an electrode (first electrode) 64 at the distal end 63 or near the distal end 63. The guide rod 62 is electrically connected to the energy source 14 via an electrical path (lead wire). The electrode 64 of the guide rod 62 is positioned closer to the bone B than the energy output section 56.

The energy output section 56 provided in each probe 102 is used as an identical electrode (second electrode).

The energy output sections 56 provided in the neighboring probes 102 are electrically connected to the energy source 14 via an identical electrical path (lead wire). Note that the electrical path of the energy output section 56 is different from the electrical path of the guide rod 62.

It is preferable that the energy output section 56 and guide rod 62 are disposed adjacent to each other. In this case, too, it is preferable that the number of probes 102 is greater than the number of guide rods 62.

In addition, if the switch 15 is pushed, a region R between the electrode 64 of the guide rod 62 and the energy output section 56, i.e. a region R in a biological tissue on a side farther than the energy output section 56, is properly denatured (coagulated). Thus, a portion of the deep part in the soft tissue T near the bone B and a part of the nerve N are properly cauterized.

In this manner, if the region R at a proper depth (e.g. a part of the soft tissue T near the bone B and a part of the nerve N) can be treated by passing high-frequency current to the proper depth of the biological tissue (e.g. a part of the soft tissue T near the bone B and a part of the nerve N), one of a bipolar system and a monopolar system that is used together with a return electrode (not shown) may properly be selected as the method of passing high-frequency current in the energy output sections 56 provided in the probes 102 of the needle section 54 and locally cauterizing a part of the soft. tissue T and a part of the nerve N.

(Second Modification)

Here, referring to FIG. 8 to FIG. 11, a cover portion 258 in relation to the insertion section 32 and end effector 34 will be described as a modification.

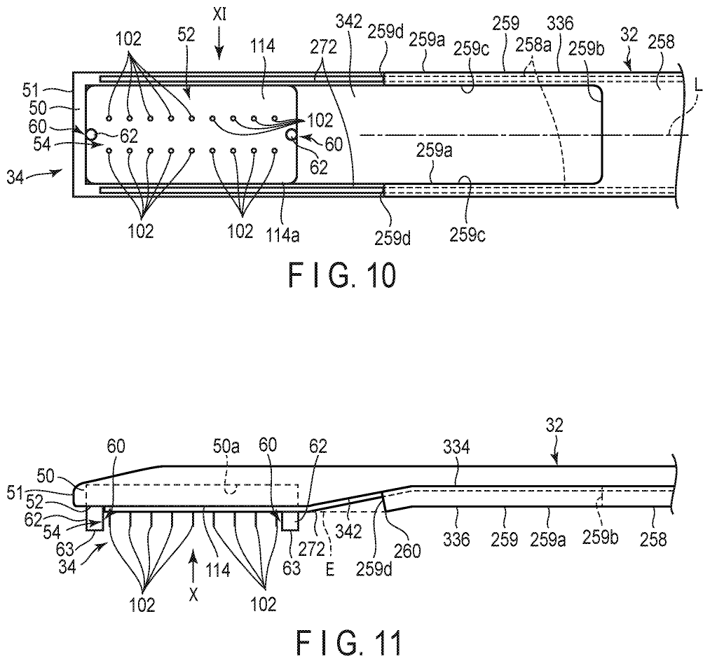

An energy treatment instrument 12 according to the present modification includes, like the first embodiment, an insertion section 32 in which a longitudinal axis L is defined, and an end effector 34. In the present modification, the treatment instrument 12 includes the cover portion 258. It is preferable that the cover portion 258 is formed of an electrically insulating resin material. The end effector 34 includes a base 52, a needle section 54, and energy output sections 56. The cover portion 258 is movable relative to the base 52. the present modification, the cover portion 258 extends along the longitudinal axis L of the insertion section 32. The cover portion 258 is movable along the longitudinal axis L of the insertion section 32. Specifically, the cover portion 258 is movable between a position illustrated in FIG. 8 and FIG. 9 and a position illustrated in FIG. 10 and FIG. 11. Thus, the cover portion 258 of the present modification differs from the cover portion 58, which is described in the first embodiment and is movable in the direction crossing the longitudinal axis L, with respect to the movable direction and the shape.

The insertion section 32 is provided with at least a. pair of electrical paths (transmission paths). The base 52 is electrically connected to the energy source 14 via lead wires (not shown) and/or a structural member or the like of the insertion section 32. For example, the insertion section 32 is electrically connected to the energy output sections 56, and may be used as a part of the transmission path for transmitting energy which is supplied from the energy source 14 to the energy output sections 56.

Guides (rails) 272 are formed on the insertion section 32 of the energy treatment instrument 12 and the housing 50 of the end effector 34 illustrated in FIG. 8 to FIG. 11. The cover portion 258 includes guides 258a which are movable along the guides 272. A distal portion 259 of the cover portion 258 is formed in a substantially U-shape. FIG. 8 and FIG. 10 illustrate an example in which the number of guides 272 of the insertion section 32 and the housing 50 of the end effector 34 is two, but the number of guides 272 may be one, or three or more. Thus, the number of guides 258a of the cover portion 258 is adjusted based on the number of guides 272 of the insertion section 32 and the housing 50 of the end effector 34.

A proximal portion (not shown) of the cover portion 258 along the longitudinal axis L of the insertion section 32 is coupled to the operation element 156 of the handle 36 shown in FIG. 1.

In addition, when the operation element 156 is advanced to the frontmost part in the slot 154, the guides 258a of the cover portion 258 move to the distal side along the longitudinal axis L along the guides 272 of the insertion section 32 and the housing 50 of the end effector 34. Thus, the distal portion 259 of the cover portion 258 is disposed on the end effector 34. A second end face (end portion) 336 (to be described later) of the cover portion 258 is disposed in a position which is equal to the tip end 104 of the needle section 54, or in a position (projection direction side) which projects from the tip end 104. This position is defined as “first position” of the cover portion 258. Thus, when the cover portion 258 is in the first position, the second end face 336 of the cover portion 258 is disposed. in a position which. is equal to the tip end 104 of the probe 102 of the needle section 54, or in a position which is farther from the defining surface 114 of the base 52 than the tip end 104. In this manner, when the cover portion 258 is in the first position, the second end face (end portion) 336 of the cover portion 258 is disposed in a position which is equal to the tip end 104 of the needle section 54, or in a position which projects from the tip end 104, thereby protecting the tip ends 104 and output sections 56 of the needle section 54. Specifically, when the cover portion 258 is in the first position, the cover portion 258 surrounds and covers the outside of the needle section 54 in such a state that the tip ends (needle points) 104 of the probes 102 are not exposed.

When the operation element 156 is retreated to the rearmost position in the slot 154, the guides 258a of the cover portion 258 move to the proximal side along the longitudinal axis L along the guides 272 of the insertion section 32 and the housing 50 of the end effector 34. The second end face 336 of the cover portion 258 is evacuated from the tip ends 104 and output sections 54 of the needle section 54. At this time, the second end face 336 of the cover portion 258 is disposed in a position (base 52 side) which is closer to the base 52 than the tip ends 104 and energy output sections 56 of the needle section 54. This position is defined as “second position” of the cover portion 258. Thus, the distal portion 259 of the cover portion 258 is evacuated from the end effector 34 and is disposed in the distal portion of the insertion section 32. Therefore, the second end face 336 of the cover portion 258 is disposed in a position which is closer to the defining surface 114 of the base 52 than the tip ends 104 and energy output sections 56 of the probes 102 of the needle section 54. Specifically, when the cover portion 258 is in the second position, the tip ends (needle points) 104 and energy output sections 56 of the probes 102 project from the second end face (end portion) 336 with respect to the cover portion 258. At this time, the tip ends (needle points) 104 and energy output sections 56 of the probes 102 are exposed from the cover portion 258. Thus, when the cover portion 258 is in the second position, the end effector 34 is capable of piercing the tip ends 104 and energy output sections 56 of the needle section 54 into the biological tissue.

In addition, the cover portion 258 can advance and retreat (can move) between the first position and the second position.

In the present modification, the distal portion 259 of the cover portion 258 includes a pair of extension portions 259a which extend straight or substantially straight, and a proximal edge 259b which is formed in proximal portions of the extension portions 259a, is perpendicular to the longitudinal axis L and faces the distal side along the longitudinal axis L. The paired extension portions 259a include a pair of opposed surfaces 259c which are opposed to each other. When the cover portion 258 is in the first position, the needle section 54 is present between the paired opposed surfaces 259c. In addition, the needle section 54 is opposed to the proximal edge 259b.

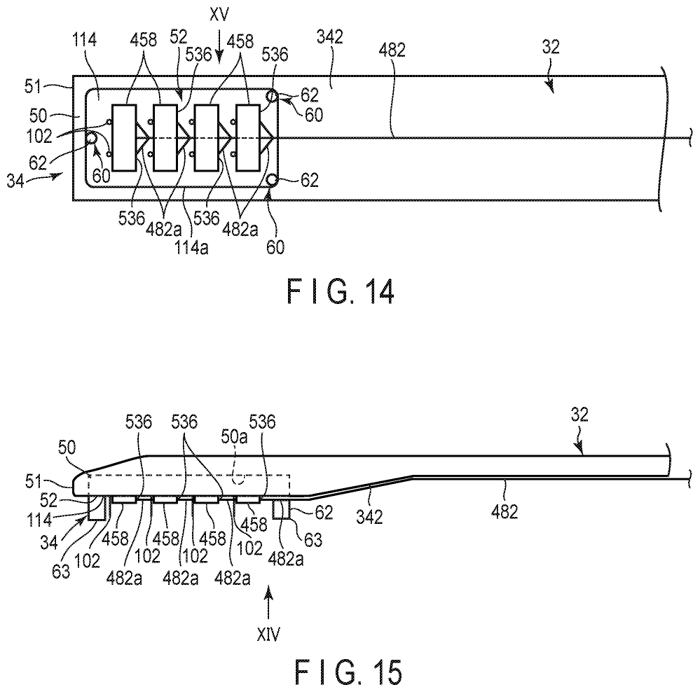

The distal portion 259 of the cover portion 258 includes a first end face 334 which is opposed to or put in contact with the inner peripheral surface 50a of the housing 50 in which the base 52 is provided; and a second end face (reference surface (reference edge)) 336 which is located opposite to the first end face 334. Like the second end face 136 described in the first embodiment, the second end face 336 is formed as an end portion. The first end face 334 and second end face 336 may be planar surfaces or curved surfaces. As illustrated in FIG. 14, the thickness of the distal portion 259 of the cover portion 258 (the distance between the first end face 334 and second end face 336) is set such that the tip ends 104 of the probes 102 are not visually recognized when the end effector 34 is observed from a lateral side. Thus, when the cover portion 258 is in the first position, the tip ends 104 of the needle section 54 are disposed in a position which is closer to the defining surface 114 of the base 52 than the second end face (reference surface) 336.

An inclined surface 342 is formed between the proximal portion of the housing 50 of the end effector 34 and the distal portion of the insertion section 32. When the cover portion 258 is disposed in the second position, an edge portion 260 at the distal end of the cover portion 258 is, for example, located on an extension line E of the guide 272 in the end effector 34.. Thus, in the case of treating a part of the soft tissue T and a part of the nerve. N1, N2, N3 by using the end effector 34, when the guides 272 are abutted on the surface of the soft tissue T, the edge portion 260 at the distal end of the cover portion 258 is prevented from interfering with the surface of the soft tissue T. Therefore, when a part of the soft tissue T and a part of the nerve N1, N2, N3 are treated by using the end effector 34, the cover portion 258, which is disposed apart from the end effector 34, scarcely becomes an obstacle.

When the position of the distal end 51 of the housing 50 and the position of distal ends 259d of the cover portion 258 agree or substantially agree along the longitudinal axis L, the position of the outer edge 114a of the defining surface 114 of the base 52 and the position of the proximal edge 259b of the cover portion 258 agree or substantially agree along the longitudinal axis L.

(Third Modification)

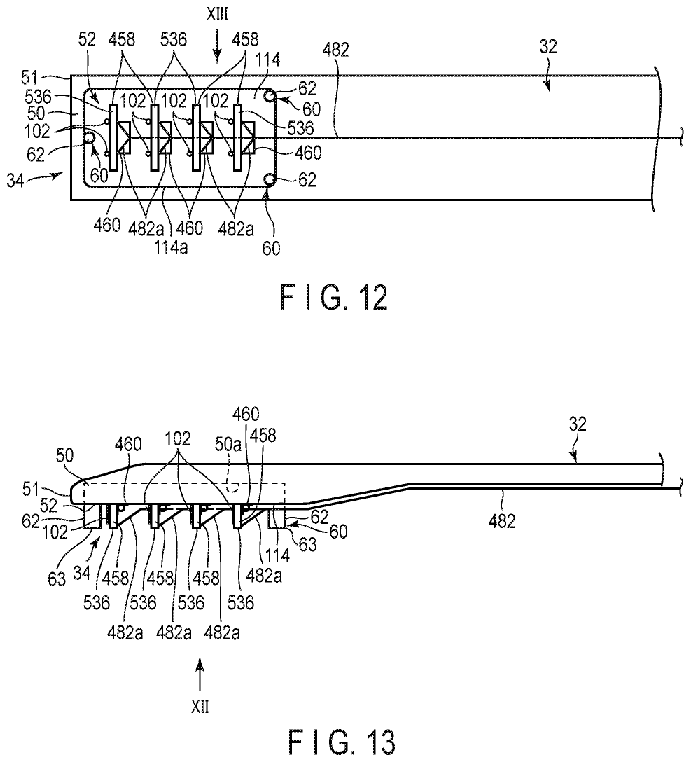

Here, referring to FIG. 12 to FIG. 15, a cover portion 458 in relation to the insertion section 32 and end effector 34 will be described as a modification.

An energy treatment instrument 12 according to the present modification includes, like the first embodiment, an insertion section 32 in which a longitudinal axis L is defined, and an end effector 34. The end effector 34 includes a base 52, a needle section 54, energy output sections 56, and a plurality of cover portions 458. The cover portions 458 are provided on the base 52 and are movable relative to the base 52. The cover portions 458 are movable between a standing position illustrated in FIG. 12 and FIG. 13 and a lying position illustrated in FIG. 14 and FIG. 15. Thus, the cover portions 458 of the present modification are different from the cover portion 58 described in the first embodiment and the cover portion 258 described in the second modification with respect to the movable direction and the shape.

It is preferable that the cover portions 458 are formed of an electrically insulating resin material. Each cover portion 458 is formed in a plate shape and, in this embodiment, is coupled to the defining surface 114 of the base 52 by a hinge 460. For the purpose of simple description, a description is given of an example in which each cover portion 458 is formed as a rectangular plate-shaped member. Each hinge 460 is rotated, for example, in a range between 0 degree and 90 degrees, or in a range less than 90 degrees.

For example, each cover portion 458 is disposed adjacent to a pair of probes 102 which are juxtaposed. Here, each cover portion 458 neighbors the proximal side of the probes 102 along the longitudinal axis L. A moving body 482, which extends to the proximal side along the longitudinal axis L of the insertion section 32, is coupled to each cover portion 458. A wire, for instance, is used as the moving body 482. A proximal end of the moving body 482 is coupled to the operation element 156 provided in the handle 36.

Each cover portion 458 is coupled via a support wire 482a to the moving body 482 which extends along the longitudinal axis L of the insertion section 32.

In addition, when the moving body 482 is positioned at the distal end along the longitudinal axis L of the slot 154 of the handle 36, the cover portions 458 are disposed in a first position illustrated in FIG. 12 and FIG. 13. In the first position illustrated in FIG. 12 and FIG. 13, the plate-shaped cover portions 458 stand, for example, in parallel to the probes 102. A reference surface (end portion) 536 (to be described later) of the cover portion 458 is disposed in a position which is equal to the tip end 104 of the needle section 54, or in a position (projection direction side) which projects from the tip end 104. Thus, when the cover portion 458 is in the first position, the reference surface 536 of the cover portion 458 is disposed in a position which is equal to the tip end 104 of the probe 102 of the needle section 54, or in a position which is farther from the defining surface 114 of the base 52 than the tip end 104. In this manner, when the cover portion 458 is in the first position, the end face (end portion) 536 of the cover portion 458 is disposed in the position which is equal to the tip end 104 of the needle section 54 or in the position which projects from the tip end 104, thereby protecting the tip ends 104 and output sections 56 of the needle section 54. Specifically, when the cover portions 458 are in the first position, the reference surface 536 of the cover portion 458 is disposed on the outside of the needle section 458 in such a state that the tip ends (needle points) 104 of the probes 102 are not exposed. At this time, each cover portion 458 collectively protects part of the probes 102.

When the moving body 482 is positioned at the proximal end along the longitudinal axis L of the slot 154 of the handle 36, each cover portion 458 is disposed in a second position illustrated in FIG. 14 and FIG. 15. The reference surface 536 of the cover portion 458 is disposed in a position (base 52 side) which is closer to the base 52 than the tip end 104 and energy output section 56 of the needle section 54. In the second position illustrated in FIG. 14 and FIG. 15, the plate-shaped cover portion 458 is fallen and laid relative to the probes 102. In this manner, when the cover portion 458 is in the second position, the end face (end portion) 536 of the cover portion 458 is evacuated from the tip end 104 and output section 56 of the needle section 54. In other words, when the cover portion 58 is in the second position, the tip ends (needle points) 104 and energy output sections 56 of the probes 102 project from the reference surface (end portion) 536 with respect to the cover portion 458. At this time, the tip ends (needle points) 104 and energy output sections 56 of the probes 102 are exposed from the cover portion 458. Thus, when the cover portions 458 are in the second position, the end effector 34 is capable of piercing the tip ends 104 and energy output sections 56 of the needle section 54 into the biological tissue. Although it is preferable that the cover portions 458 are moved at the same time, the cover portions 458 may be moved with time differences.

In addition, the cover portion 458 can advance and retreat (can move) between the first position and the second position.

The cover portion 458 includes the reference surface (reference edge) 536. The reference surface 536 is formed as an end portion, like the second end face 136 described in the first embodiment and the second end face 336 described in the second embodiment. The reference surface 536 may be a planar surface or a curved surface. Here, the height of the reference surface 536 of the cover portion 458 relative to the defining surface 114 of the base 52 is equal to or greater than the distance of the tip end 104 of the probe 102 from the defining surface 114 of the base 52. Thus, when the cover portions 458 are disposed in the position illustrated in FIG. 12 and FIG. 13, the passage from the entrance to the treatment target toward the treatment target is protected from the tip end 104 of each probe 102, and each probe 102 for use in a treatment is protected.

It is preferable that in the cover portion 458, a torsion spring (not shown) is provided as an urging body 74 for the hinge 460. In this case, the cover portion 458 is urged to the first position illustrated in FIG. 12 and FIG. 13.

As described above in the first embodiment, the second modification and the third modification, the movable directions of the cover portions 58, 258 and 458 are different from each other. The example illustrated in FIG. 5 and the method described in the first modification can be used for the energy output sections 56 provided in the probes 102 in the second modification and the third modification. This is similarly applicable to a second embodiment and a third embodiment which will be described below.

### Second Embodiment

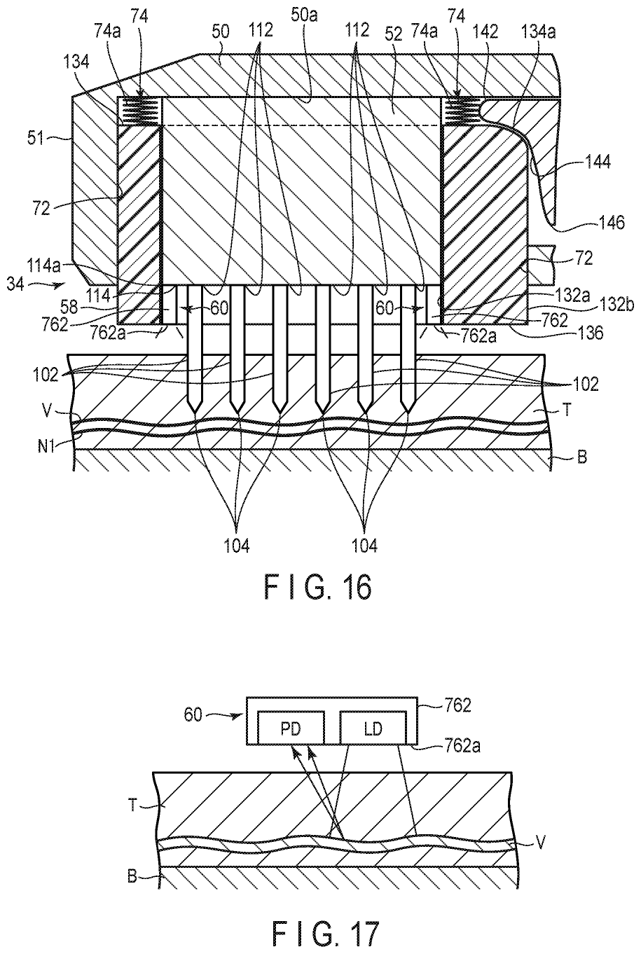

Next, a second embodiment will be described with reference to FIG. 16 and FIG. 17. This embodiment is a modification of the first embodiment including each modification. The same members or the members having the same functions as the members described in the first embodiment are denoted by like reference signs, and a detailed description thereof is omitted.

As illustrated in FIG. 16, the end effector 34 of the present embodiment includes a detector (sensing portion) 60 which detects the presence of an organ such as a bone, blood vessel, nerve, etc. which is located farther than the tip end (end portion) 104 of the probe 102, when the tip ends 104 of the probes 102 of the needle section 54 are pierced into a soft tissue (biological tissue) T. Thus, when the probes 102 are pierced into the soft tissue (biological tissue) T, the detector (sensing portion) 60 detects the position. of the organ. which is located farther than the tip end (end portion) 104 of the probe 102. The detector 60 includes one, or a plurality of, sensors 762 which detect the presence of the organ. by waves such as light, ultrasonic vibration, etc. For example, it is assumed that the number of sensors 762 is two. The sensors 762 are electrically connected to the energy source 14 via the base 52.

The sensors 762 are juxtaposed with the probes 102. In the present embodiment, an example is illustrated. in which the sensors 762 are disposed in an outside region of the needle section 54. However, the sensors 762 may be disposed in an inside region of the needle section 54, for example, among the probes 102.

In the present embodiment, a description is given of an example in which an optical sensor is used as the sensor 762, the optical sensor optically detecting the presence of an organ, in particular, a blood vessel, from the presence/absence of a blood flow.

The system 10 according to the present embodiment includes an additional switch (not shown) in addition to the switch 15 described in the first embodiment. It is preferable that the additional switch is provided on the treatment instrument 12.

In the present embodiment, a description is given on the assumption that the detector 60 includes two optical sensors 762. However, the number of optical sensors 762 may not be plural, such as two, but may be only one.

As illustrated in. FIG. 17, the optical sensor 762 includes, for example, a laser diode LD and a photodiode PD. The laser diode LD radiates light, such as infrared, toward the bone B through the soft tissue T. The photodiode PD receives reflective light by the radiation. Based on a Doppler phenomenon of light, the energy source 14 analyzes a frequency spectrum of a detected signal, and acquires information relating to the flow of blood. Accordingly, the position of the blood vessel V is detected by the optical sensor 762.

At this time, the energy source 14 recognizes a distance to the blood vessel V, based on the magnitude of the detected signal, by using the optical sensor 762. Thus, the surgeon can understand the distance of the blood vessel V relative to the optical sensor 762.

When the second end face 136 of the cover portion 58 is in contact with the surface of the soft tissue T, an end face 762a of the optical sensor 762 may come in contact with the surface of the soft tissue T. Also when the end face 762a of the optical sensor 762 is in contact with the surface of the soft tissue T, the energy source 14 can recognize the distance to the blood vessel V, based on the magnitude of the detected signal, by using the optical sensor 762.

Here, as illustrated in FIG. 16, it is understood that the blood vessel V and nerve N exist near the surface of the bone B. In addition, the distances of the blood vessel V and nerve N relative to the surface of the bone B are substantially equal.

As illustrated in FIG. 16, when the cover portion 58 is in the second position, the end face 136 of the cover portion 58 and the end face 762a of the sensor 762 are disposed to be substantially flush with each other. In addition, a distance of the tip end 104 of the probe 102 relative to the second end face 136 of the cover portion 58, i.e. the projection length of the probe 102, is understood. Thus, the surgeon can dispose the tip ends 104 of the probes 102 in contact with, or in close proximity to, the blood vessel V and nerve N, by setting the distance between the end face 762a of the optical sensor 762 and the blood vessel V to be equal to the projection length of the probes 102.

Thus, the energy output sections 56 provided in the probes 102 can be disposed near the nerve N extending near the surface of the bone B. Accordingly, the nerve V can more effectively be treated. In this manner, by using the energy treatment instrument 12 of the present embodiment, the surgeon can easily apply energy to a proper depth. Therefore, according to the present embodiment, there can be provided the energy treatment instrument 12 which can easily recognize the presence of a biological tissue other than a treatment target to which energy is output.

In the present embodiment, the description was given of the example in which the cover portion 58 described in the first embodiment is used. However, the cover portion 258 described in the second modification or the cover portion 458 described in the third modification may be used as appropriate. Further, the cover portion 58 is not necessarily required. Here, although the second end face 136 of the cover portion 58 was described as the reference surface, the position of the blood vessel V and nerve N may be estimated by using, for example, the defining surface 14 of the base 52 as the reference surface.

The optical sensor 762 may adopt various measuring methods using light as waves, such as triangulation, optical coherence tomography (OCT), or Tod', aside from the example in which the Doppler phenomenon of light is used.

In the present embodiment, the example was described in which the optical sensor 762 is used. However, in place of the optical sensor, or together with the optical sensor, a reflection-type ultrasonic sensor using, for example, ultrasonic vibration as waves may be used, and the position of, for example, the bone B may be estimated. It is understood that the position of the nerve N relative to the bone B exists in a proper range, although the position varies from patient to patient. Thus, as well as the optical sensor, an ultrasonic sensor can be used as the sensor 762. Specifically, the detector 60 may include an ultrasonic sensor which detects the presence of an organ, such as the bone B, by inputting ultrasonic vibration to a biological tissue in the state in which the probes 102 are pierced into the biological tissue.

In the present embodiment, the description was given of the example in which the cover portion 58 described in the first embodiment is used. However, the cover portion 58 is not necessarily required. Thus, it is not always necessary that the second end face 136 of the cover portion 58 is put in contact with the surface of the soft tissue T. On the other hand, the treatment instrument 12 according to the present embodiment may use, as appropriate, the cover portion 258 described in the second modification of the first embodiment or the cover portion 458 described in the third modification of the first embodiment.

### Third Embodiment

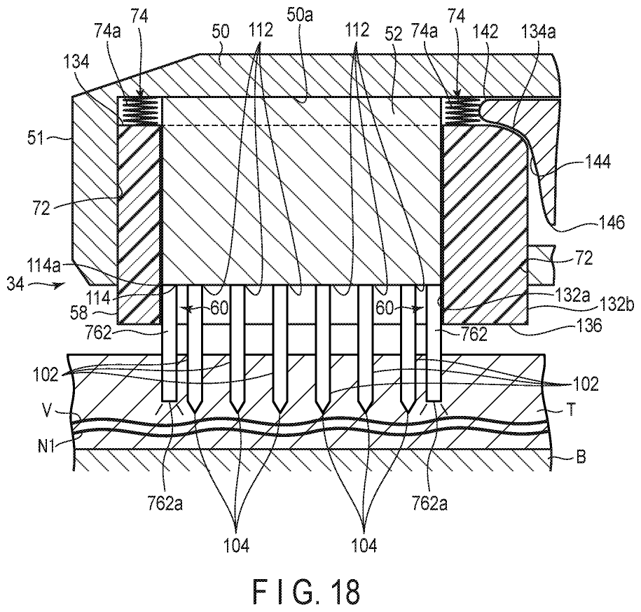

Next, a third embodiment will be described with reference to FIG. 18. This embodiment is a modification of the first embodiment including each modification and the second embodiment. The same members or the members having the same functions as the members described in the first embodiment and the second embodiment are denoted by like reference signs, and a detailed description thereof is omitted. The present embodiment is, especially, a modification of the second embodiment.

The example was described in which the detector 60 (sensor 762) of the second embodiment estimates the distance to a specific organ, such as the blood vessel V or bone B, from a position separate from a tissue or from the surface of the tissue. An example will be described in which the detector 60 (sensor 762) of the present embodiment estimates the distance to a specific organ, such as the blood vessel V or bone B, from the inside of a tissue.

As illustrated in FIG. 18, when the cover portion 58 is in the second position, the end face 762a of the sensor 762 projects more toward the tip end 104 of the probe 102 than the second end face 136 of the cover portion 58. On the other hand, the end face 762a of the sensor 762 is positioned closer to the defining surface 114 of the base 52 than the tip end 104 of the probe 102.

The sensor 762 is pierced into the biological tissue. Thus, when an optical sensor is used as the sensor 762, for example, a blood flow can be measured with high sensitivity, and the position of the blood vessel can be recognized. In addition, when ultrasonic vibration is used as the sensor 762, for example, the position of the bone B can be measured with high sensitivity. Accordingly, the energy output section 56 can surely be disposed in close proximity to, or in contact with, the position of the nerve N of the treatment target near the surface of the bone B.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An energy treatment instrument comprising:
an insertion section in which a longitudinal axis is defined;
a probe provided on a distal side along the longitudinal axis of the insertion section and configured to be pierced into a biological tissue;
an output section provided on the probe and configured to output energy to an outside of the probe when supplied with energy; and
a detector configured to recognize presence of an organ which is located farther than an end portion of the probe when the probe is pierced into the biological tissue.

2. The energy treatment instrument of claim 1, wherein:
the detector includes a guide rod which is juxtaposed with the probe, and
the guide rod includes a distal end which is abuttable on a bone as one of the organ.

3. The energy treatment instrument of claim 1, wherein the detector includes a sensor configured to detect the presence of a blood vessel, a nerve or a bone as one of the organ by waves.

4. The energy treatment instrument of claim 1, wherein the detector includes an optical Sensor configured to optically detect the presence of a blood vessel, a nerve or a bone as one of the organ.

5. energy treatment instrument of claim 4, wherein the optical sensor is juxtaposed with the probe.

6. The energy treatment instrument of claim 4, wherein when the end portion of the probe is positioned in contact with, or in close proximity to, the blood vessel, the nerve or the bone, the optical sensor is located outside the biological tissue.

7. The energy treatment instrument of claim 1, wherein the detector includes an ultrasonic sensor configured to detect the presence of a blood vessel, a nerve or a bone as one of the organ by inputting ultrasonic vibration to the biological tissue in a state in which the probe is pierced into the biological tissue.

8. The energy treatment instrument of claim 1, wherein the output section is provided on the end portion of the probe.

9. An energy treatment instrument comprising:
an insertion section in which a longitudinal axis is defined;
a base provided on a distal side along the longitudinal axis of the insertion section;
a probe including an end portion which projects relative to the base, and configured to be pierced into a biological tissue from the end portion;
an output section provided on the end portion of the probe and configured to output energy to an outside of the probe when supplied with energy; and
a guide rod projecting relative to the base in a state in which the guide is juxtaposed with the probe, the guide rod including a distal end which is farther from the base than the end portion of the probe, and the guide rod being abuttable on a hard tissue.

10. An energy treatment instrument comprising:
an insertion section in which a longitudinal axis is defined;
a base provided on a distal side along the longitudinal axis of the insertion section;
a probe including an end portion which projects relative to the base, and configured to be pierced into a biological tissue from the end portion;
an output section provided on the probe and configured to output energy to an outside of the probe when supplied with energy; and
a detector configured to detect presence of an organ which is located farther than the end portion of the probe when the probe is pierced into the biological tissue.

11. The energy treatment instrument of claim 10, wherein the detector includes a sensor configured to detect the presence of the organ by waves.

12. The energy treatment instrument of claim 10, wherein the detector includes an optical sensor configured to optically detect presence of a blood vessel, a nerve or a bone as one of the organs.

13. The energy treatment instrument of claim 12, wherein the optical sensor is juxtaposed with the probe.

14. The energy treatment instrument of claim 12, wherein when the end portion of the probe is positioned in contact with, or in close proximity to, the blood vessel, the nerve or the bone, the optical sensor is located outside the biological tissue.
